(19) United States
(12) Patent Application Publication
(10) Pub. No.: US 2026/0275177 A1
(43) Pub. Date: Sep. 17, 2026

(54) PROTEIN-CONTAINING ADHESIVE COMPOSITIONS

(71) Applicant: TANDEM REPEAT TECHNOLOGIES, INC., Philadelphia, PA (US)

(72) Inventor: Benjamin D. Allen, Beverly, MA (US)

(21) Appl. No.: 19/471,841

(22) PCT Filed: Apr. 3, 2024
(86) PCT No.: PCT/US2024/022757

§ 371 Date: Oct. 2, 2025

Related U.S. Application Data
(60) Provisional application No. 63/494,249, filed on Apr. 5, 2023.

(51) Int. Cl. C09J 189/00; C08K 3/16; C08K 3/22; C08K 5/05; C08K 5/053; C08K 5/21; C09J 11/04; C09J 11/06

(52) U.S. Cl. CPC C09J 189/00; C09J 11/04; C09J 11/06; C08K 3/16; C08K 3/22; C08K 5/05; C08K 5/053; C08K 5/21

(57) ABSTRACT

In one aspect, the disclosure relates to protein- and peptide-based adhesive compositions, methods of making the same, substrates and articles comprising the same, and methods of adhering substrates using the same. In one aspect, the adhesive composition includes one or more polypeptides, a solvent, a denaturing agent, and a plasticizer. The adhesive compositions have a high peel-strength and exhibit self-healing properties. This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/494,249 filed on Apr. 5, 2023, which is incorporated herein by reference in its entirety.

### CROSS-REFERENCE TO SEQUENCE LISTING

This application contains a sequence listing filed in ST.26 format entitled “320020_2020_Sequence_Listing.xml” created on Mar. 31, 2023, and having a file size of 211 KB. The content of the sequence listing is incorporated herein in its entirety.

### BACKGROUND

Adhesives are critical components of a variety of manufactured goods including garments, packaging, electronics, and composite materials. However, many common adhesives are derived from non-renewable petroleum products, are not recyclable or biodegradable, and/or bond irreversibly. Furthermore, when adhered surfaces are debonded or otherwise damaged, many common adhesives, once set, are incapable of self-healing or readhesion, thereby limiting the lifetimes of adhered materials and articles constructed from the same.

Adhesives are required in many textile products and impart features including durable and, in some cases, leak-tight joins as well as enhancing the aesthetic properties of the textiles. Textiles useful in consumer and industrial settings may have many different properties, however, including structure (e.g. knit, woven, nonwoven), chemical makeup (e.g. synthetic, protein, or cellulose fiber), thickness, flexibility, drape, and the like. These properties and others of the substrates to be adhered to one another must be studied and a specific adhesive chosen for a given application.

Protein materials are ubiquitous in nature, playing critical protective and structural roles in forms as familiar as our own skin, hair, and fingernails, as well as providing the basis for some of our oldest technologies: fibers and textiles based on animal-derived materials like silk and wool. The development of modern biotechnology offers new possibilities for protein materials, including genetic engineering of a wide array of material properties, intrinsic biocompatibility and biodegradability, and sustainable, animal-free production in recombinant microbes. The most mature recombinant technology for protein-material production has been achieved for sequences based on various types of silk.

Recombinant silk-based sequences have been produced at scale and manufactured into a variety of products, including blended textiles, cosmetic additives, and coatings. However, silk-based sequences suffer from numerous drawbacks, including high molecular weights that stymie high-titer production, the difficulty of thermal manufacturing, and the limited tunability of mechanical properties. The recent introduction of recombinant materials based on squid-ring teeth (SRT) sequences has offered improvements to silk-based sequences, including lower molecular weight that enables high-titer production and simpler gene construction, thermal processability, and straightforward genetic tuning of mechanical properties. In addition to these benefits, SRT sequences demonstrate behaviors not observed in silks, including self-healing under mild conditions and directed self-assembly of non-biological materials into ordered nanomaterial composites.

Although protein-containing adhesives such as casein and hide glue have long been known, these glues may not be suitable for applications to all desired substrates and may have unsuitable properties including a short open time, lack of water resistance, unpleasant smell, animal origin, and the like. Plant-based adhesives have also been developed from proteins in the seeds of crop plants, including wheat, soy, cotton, canola, and maize. However, these proteins suffer from lower resistance to dissolution and hydrolysis in water, and chemical crosslinking is required for acceptable performance in many applications. An ideal protein- or peptide-based adhesive would take advantage of self-healing and self-assembly capabilities of the protein or peptide to promote better adhesion between substrates, allow for repair of damage, and provide finished articles incorporating the adhesive such as textiles having desirable properties.

Despite advances in adhesives research, there is still a scarcity of adhesive compositions that are effective at bonding varied textile and other surfaces to create durable layered structures and joins, are capable of at least some degree of self-healing, have a high peel strength, and are also biodegradable and/or recyclable. These needs and other needs are satisfied by the present disclosure.

### SUMMARY

In accordance with the purpose(s) of the present disclosure, as embodied and broadly described herein, the disclosure, in one aspect, relates to protein- and peptide-based adhesive compositions, methods of making the same, substrates and articles comprising the same, and methods of adhering substrates using the same.

In one aspect, the adhesive composition includes one or more polypeptides, a solvent, a denaturing agent, and a plasticizer. In an aspect, the one or more polypeptides include at least one peptide having Formula I:

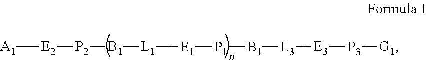

wherein
A₁ is absent, is a methionine, or is an amino acid sequence 1 to 4 residues in length;
E₁ a GLY-rich amino acid sequence 8 to 58 residues in length including amino acids selected from the group consisting of glycine, leucine, tyrosine, phenylalanine, and proline, or any combination thereof;
B₁ is an ASTVH-rich sequence amino acid sequence 6 to 17 residues in length including amino acids selected from the group consisting of alanine, serine, threonine, valine, histidine, glycine, glutamine, and proline, or any combination thereof.
L₁ is absent or is an amino acid sequence 1 to 7 residues in length including amino acids selected from the group consisting of proline, glycine, leucine, serine, and threonine, or any combination thereof;
E₂ is absent or is E₁;
E₃ is absent or is E₁;
P₁ is absent or is proline;
P₂ is absent or is P₁;
P₃ is absent or is P₁;
L₃ is absent or is L₁;
G₁ is absent or is an amino acid sequence 1 to 4 residues in length; and
wherein n is 4 to 100.

In any of these aspects, the one or more polypeptides can be synthetic or recombinant supramolecular polypeptides or can be at least two distinct polypeptides.

Also disclosed herein are methods of making the disclosed compositions and methods of bonding two substrates together using the compositions and adhesive films produced therefrom. In one aspect, the substrate can be a textile, cloth, fabric, leather, paper, plastic, an electronics component, metal, or wood.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the geometry of test samples comprising the disclosed adhesives for assessing peel strength. Two pieces of untreated cotton cloth, 4 in by 6 in, are bonded together using a 4 in by 4 in area of adhesive at one end. Three center-cut, 1 in-thick strips are cut from the sample and tested to give peel strength measurements in triplicate.

FIG. 2 shows the orientation of layers during adhesive transfer and bonding for the disclosed adhesives according to one embodiment. Adhesives to be transferred or bonded are put into contact with layers of cloth, sandwiched between layers of foil, and subjected to a heat-press procedure where heat and pressure are applied from above.

FIG. 3 shows dependence of peel strength on bonding temperature. Three-layer adhesive samples were used to bond two pieces of untreated cotton cloth at various bonding temperatures. Average peel strengths (kN/m) are based on three 1 in-strips cut from a single 4 in by 6 in bonded sample as shown in FIG. 1. Error bars represent the standard error of the mean from the triplicate measurement.

FIG. 4 shows dependence of peel strength on number of adhesive layers. One or more adhesive layers were used to bond two pieces of untreated cotton cloth at either 157° C. (black circles) or 129° C. (grey circles). Average peel strengths (kN/m) are based on three 1 in-strips cut from a single 4 in by 6 in bonded sample as shown in FIG. 1. Error bars represent the standard error of the mean from the triplicate measurement.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are adhesive compositions that are effective at bonding various types of textile surfaces and other substrates to create durable layered structures and joins, are capable of self-healing, have a high peel strength, and that are biodegradable and/or recyclable.

### Adhesive Compositions

In one aspect, the adhesive composition includes one or more polypeptides, a solvent, a denaturing agent, and a plasticizer. In an aspect, the one or more polypeptides include at least one peptide having Formula I:

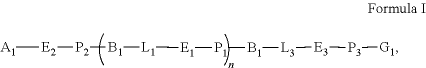

wherein
A₁ is absent, is a methionine, or is an amino acid sequence 1 to 4 residues in length;
E₁ a GLY-rich amino acid sequence 8 to 58 residues in length including amino acids selected from the group consisting of glycine, leucine, tyrosine, phenylalanine, and proline, or any combination thereof;
B₁ is an ASTVH-rich sequence amino acid sequence 6 to 17 residues in length including amino acids selected from the group consisting of alanine, serine, threonine, valine, histidine, glycine, glutamine, and proline, or any combination thereof.
L₁ is absent or is an amino acid sequence 1 to 7 residues in length including amino acids selected from the group consisting of proline, glycine, leucine, serine, and threonine, or any combination thereof;
E₂ is absent or is E₁;
E₃ is absent or is E₁;
P₁ is absent or is proline;
P₂ is absent or is P₁;
P₃ is absent or is P₁;
L₃ is absent or is L₁;
G₁ is absent or is an amino acid sequence 1 to 4 residues in length; and
wherein n is 4 to 100.

In various, non-limiting embodiments, E₂ and E₃ can be absent and L₃ can also be absent, or E₂ and E₃ can have the same sequence as E₁, while L₃ can have the same sequence as L₁. In any of these aspects, the one or more polypeptides can be synthetic or recombinant supramolecular polypeptides. In another aspect, the one or more polypeptides can be or include at least two distinct polypeptides.

In one aspect, the one or more polypeptides can be present in the adhesive composition in an amount of from about 0.01 to about 50% w/v, or from about 0.05 to about 40% w/v, from about 1 to about 25% w/v, from about 5 to about 20% w/v, from about, from about 10 to about 25% w/v, or from about 1 to about 10% w/v.

In one aspect, the ASTVH-rich sequence includes at least one alanine, at least one serine, at least one threonine, at least one valine, and at least one histidine. In another aspect, the ASTVH-rich sequence can be selected from any one of SEQ ID NOs. 1-103.

The term ASTVH-rich sequence refers to a sequence that can comprise additional sequences and in a different order than a peptide of ASTVH. For example, in some embodiments, the ASTVH-rich sequence includes at least one alanine, at least one serine, at least one threonine, at least one valine, and at least one histidine. In some embodiments, the ASTVH-rich sequence includes two or more alanines. In some embodiments, the ASTVH-rich sequence includes two or more serines. In some embodiments, the ASTVH-rich sequence includes two or more threonines. In some embodiments, the ASTVH-rich sequence includes two or more valines. In some embodiments, the ASTVH-rich sequence includes two or more histidines.

In another aspect, L₁ can be selected from any one of SEQ ID NOs. 236-242, or can be PST, PS, P, ST, or S. In some embodiments, L₁ is absent or is an amino acid sequence 1 to 7 residues in length including amino acids selected from the group consisting of glycine, leucine, serine, and threonine, or any combination thereof. In some embodiments, L₁ is absent.

In still another aspect, the GLY-rich sequence can include at least one glycine, at least one leucine, and at least one tyrosine. In another aspect, the GLY-rich sequence can be selected from any one of SEQ ID NOs. 119-235. In some embodiments, E₁ is an GLY-rich amino acid sequence 8 to 58 residues in length comprising amino acids selected from the group consisting of glycine, leucine, tyrosine, phenylalanine, and proline, or any combination thereof. In some embodiments, E₁ is a third amino sequence comprising a combination of two or more of glycine, leucine, tyrosine, phenylalanine, and proline.

In some embodiments, G₁ is absent or is an amino acid sequence 1 to 4 residues in length.

In some embodiments, G₁ is an amino acid sequence including serine and/or threonine. In some embodiments, G₁ is absent.

In some embodiments, n is a range between 4-100, 4-90, 4-80, 4-70, 4-60, 4-50, 4-40, 4-30, 4-20, 4-10, 6-20, 8-20, 10-20, 10-30, 4-16, 6-16, 8-16, 10-16, 12-16, 4-12, 6-12, 8-12, or 10-12.

In some embodiments, Formula I can be selected from any one of SEQ ID NOs. 104-118.

Numerous solvents are contemplated in the adhesive compositions described herein.

Non-limiting examples include, but are not limited to, DMSO, aqueous formic acid, neat formic acid, aqueous solutions of ammonia, an alkylamine, hexafluoroisopropanol (HFIP), or any combination thereof. In one aspect, when the adhesive compositions include an alkylamine solvent, the alkylamine can be selected from triethylamine, diethylamine, ethylamine, or any combination thereof. In one preferred embodiment, the solvent is HFIP. In one aspect, the solvent is present in the adhesive composition in an amount of from about 70 to about 99.9% v/v, or from about 70 to about 95% v/v, from about 70 to about 80% v/v, or from about 80 to about 90% v/v.

In another aspect, numerous denaturing agents are contemplated in the adhesive compositions described herein. In one aspect, the denaturing agent can be selected from an acid, an alkali, a salt, urea, an alcohol, a guanidinium salt, or any combination thereof. In a further aspect, when the denaturing agent is or includes an acid, the acid can be hydrochloric acid, while when the denaturing agent is or includes an alkali, the alkali can be selected from sodium hydroxide, potassium hydroxide, or any combination thereof. Salts useful as denaturing agents or components of denaturing agents include, but are not limited to, calcium chloride, lithium chloride, and combinations thereof, while useful alcohols include ethanol. In one preferred embodiment, the denaturing agent is urea. In an aspect, the denaturing agent is present in the adhesive composition in an amount of from about 0.01 to about 50% w/v, or from about 0.05 to about 40% w/v, from about 1 to about 25% w/v, or from about 1 to about 10% w/v.

In still another aspect, numerous plasticizers are contemplated in the adhesive compositions disclosed herein. In a further aspect, the plasticizer can be selected from water, glycerol, 1,4-butanediol, dibutyl tartrate, dibutyl phthalate, lactic acid, octanoic acid, palmitic acid, sorbitol, sucrose, diacetyl tartaric acid ester of monodiglycerides, or any combination thereof. In one preferred embodiment, the plasticizer is glycerol. In another aspect, the plasticizer is present in the adhesive composition in an amount of from about 0.001 to about 30% v/v, or from about 0.01 to about 25% v/v, from about 0. To about 15% v/v, or from about 5 to about 10% v/v.

The adhesive compositions disclosed herein can be provided in a variety of formats. In one aspect, the composition can be in the form of a film, a gel, a bandage, a nanotube, a micropattern, a ribbon, a colloid, a lithographic pattern, or a strip. In any of these aspects, the adhesive composition can be transparent, either prior to application to surface or after applying to a substrate. Further in this aspect, the adhesive composition can be transparent when dry.

In one aspect, the one or more polypeptides exhibit self-healing behavior, wherein “self-healing” can refer to repair of damage such as, for example, a cohesive-failure type of delamination, wherein the adhesive stays attached to each piece of fabric but rips apart from itself. Further in this aspect, re-lamination can be accomplished by application of one or more of heat, water, and pressure. In an aspect, and without wishing to be bound by theory, self-healing could proceed by re-entanglement and re-crystallization of protein chains across the boundary of the break, reforming the adhesive and rebonding the previously adhered surfaces.

### Methods of Making and Using the Adhesive Compositions

Also disclosed herein are methods of making the disclosed compositions, the methods including at least the step of admixing the one or more polypeptides, the denaturing agent, the solvent, and the plasticizer to form a mixture; and incubating the mixture for at least 12 hours to form the adhesive composition.

Furthermore, disclosed herein is a method for making an adhesive-coated substrate, the method including at least the step of applying a disclosed adhesive composition to a substrate. In one aspect, the method further includes the step of allowing the solvent to evaporate for at least 20 minutes after applying the mixture to the substrate. In another aspect, the method can include repeating the method one or more times to form an adhesive-coated substrate that includes multiple layers of adhesive. Also disclosed are adhesive films produced by the disclosed methods.

In some aspects, the substrate can be silicone-coated paper, such as that intended for a backing for a tape, peel-and-stick adhesive label, or the like.

In addition, disclosed herein is a method for bonding an adhesive film to a substrate, the method including at least the step of contacting the adhesive film with a substrate and applying pressure to the adhesive film and the substrate. In aspects where the adhesive film is provided on a silicone-coated paper, the method can further include removing the silicone-coated paper following bonding the adhesive film to the substrate.

In any of these aspects, a heat press can be used to facilitate bonding of the adhesive film to the substrate. In one aspect, when a heat press is used, the heat press subjects the substrate and the adhesive film to a temperature of from about 60° C. to about 200° C., or from about 140° C. to about 190° C., from about 60° C. to about 100° C. In any of these aspects, the maximum temperature setting of the heat press can be chosen by the operator based on the nature of the substrate; i.e. a synthetic polymeric textile substrate may not be raised to the same temperature as a cotton textile substrate.

In one aspect, the substrate can be a textile, cloth, fabric, leather, paper, plastic, an electronics component, metal, or wood. In one aspect, when the substrate is a textile, the textile can be a cellulosic textile, a synthetic textile, a protein-based textile, or any combination thereof.

In one aspect, the textile can be a woven textile, a knit textile, a non-woven textile, or any combination thereof. In a further aspect, when the textiles are cellulosic, the textiles can be or include cotton, linen, bamboo, or any combination thereof; when the textiles are synthetic, the textiles can be or include polyester, acrylic, nylon, or any combination thereof; and when the textiles are protein-based textiles, the textiles can be or include wool, silk, or any combination thereof.

In another aspect, a first and a second substrate can be bonded together using an adhesive film adhered to the first substrate as disclosed herein according to the following method:
applying pressure to the adhesive film and first substrate, thereby bonding the adhesive film to the first substrate;
contacting the adhesive film with a second substrate in a desired orientation; and
applying pressure to the first substrate, the adhesive film, and the second substrate.

In an aspect, the first and second substrates can be the same, such as, for example, the same type of textile, or can be different, such as, for example, a woven cotton textile and a knit silk textile.

Further in this aspect, the adhesive film can be provided on a silicone-coated paper and the method includes removing the silicone-coated paper from the first substrate prior to contacting the second substrate with the adhesive film. In another aspect, bonding a first substrate and a second substrate together can be accomplished using a heat press as described previously.

In one aspect, the first substrate and the second substrate can be textiles and the adhesive film has a peel strength of from about 0.1 to about 0.8 kN/m, or from about 0.1 to about 0.4 kN/m, from about 0.4 to about 0.8 kN/m, or about 0.25 to about 0.65 kN/m.

In any of the above aspects, the adhesive film can have a thickness of from about 25 μm to about 300 μm, or from about 100 to about 250, about 250 to about 300, about 50 to about 100, or about 175 to about 275 μm.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a solvent,” “a denaturing agent,” or “a plasticizer,” include, but are not limited to, mixtures or combinations of two or more such solvents, denaturing agents, or plasticizers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a plasticizer refers to an amount that is sufficient to achieve the desired improvement in the property modulated by the formulation component, e.g. achieving the desired level of flexibility for an adhesive formulation. The specific level in terms of wt % in a composition required as an effective amount will depend upon a variety of factors including the amount and type of protein component, amount and type of solvent, amount and type of denaturing agent, and nature of the substrates to be adhered to one another using the formulations.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Preparation of Adhesive Films

To produce an adhesive layer than can be transferred to a substrate and used for bonding, a variety of methods can be employed including extrusion and spray-coating. One such method includes a solution-evaporation method. Various solvents can be used for this purpose, including but not limited to dimethyl sulfoxide, aqueous and neat formic acid, and aqueous solutions of ammonia and its derivatives. In this example, the solvent 1,1,1,3,3,3-hexafluoroisopropanol (HFIP) has been used. To prepare a solution for casting of adhesive films, the following were combined: 6 g of TR8n8 polypeptide (SEQ ID NO. 118) powder, 2.4 g urea, and 840 μL glycerol, with the total volume brought to 30 mL with HFIP. The solution was thoroughly mixed and allowed to incubate at room temperature for at least 12 hours, at which time a uniform solution formed.

Ito spread the adhesive solution onto a substrate for solvent evaporation and film formation, an 8.5 in by 11 in sheet of silicone-coated paper substrate, or any other thin substrate with desirable release properties, was affixed to a hard, flat surface. Then, 5 mL of the adhesive solution was transferred to form a line near one end of the substrate and a draw-down coating rod (RD Specialties ½ in×16 in Formed Lab Rod, groove size #50, or any other suitable drawdown rod) was used to spread the solution evenly across the substrate. Then, the solvent was allowed to evaporate for at least 20 minutes to yield an adhesive film coated onto the release substrate.

### Example 2: Bonding Cloth Pieces Using Adhesive Film

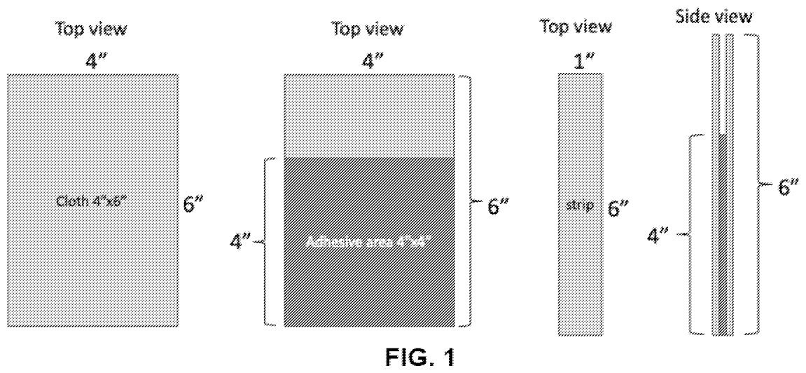

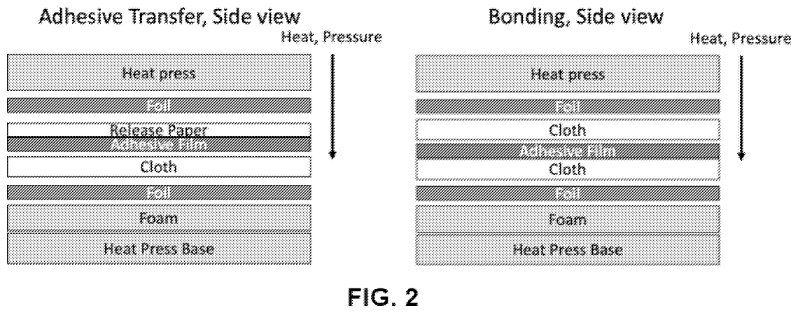

To test the peel strength yielded by various adhesive formulations and bonding parameters, bonded textile samples of standardized geometry were prepared (FIG. 1). Transfer of adhesive films from release paper onto cloth and bonding of cloth samples together were both performed using a heat-press machine (in this example, a GOLDORO Power Heat Press Machine, 15 in×15 in). To transfer adhesive film from release paper onto one piece of cloth in a first step, a 4 in by 4 in square of adhesive on release paper was placed, adhesive-side down, onto one end of a 4 in by 6 in cloth substrate. This assembly was sandwiched between two sheets of aluminum foil and placed in the heat press on a layer of polyurethane foam (FIG. 2). The adhesive film was transferred to the cloth by applying the highest available pressure with a temperature of 66° C. for 10 seconds. After this treatment, the release paper was peeled off to yield one piece of cloth coated with adhesive film. To prepare multi-layer adhesive coatings, this procedure was repeated with additional 4 in by 4 in squares of release-paper-backed adhesive film transferred onto adhesive-coated cloth instead of bare cloth.

A similar heat-press procedure was used for bonding two pieces of cloth in a second step. Onto a first piece of cloth bearing adhesive film prepared as previously described, a second piece of cloth was placed. This assembly was sandwiched between two sheets of aluminum foil and placed in the heat press on a layer of polyurethane foam (FIG. 2). Bonding of the two pieces of cloth with adhesive was accomplished by applying the highest available pressure with a temperature between 100-200° C. for 120 seconds.

### Example 3: Peel Strength Testing

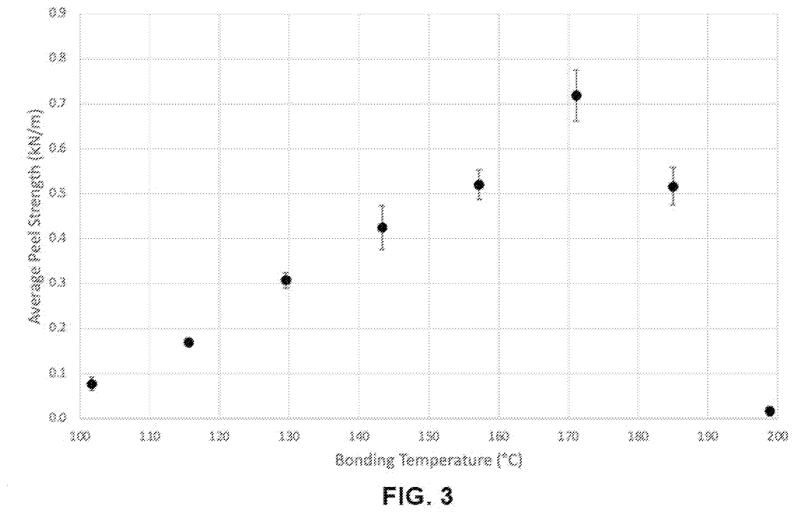

To determine appropriate parameters for bonding cloth using the adhesive formulation of this invention, bonding was attempted at a variety of different temperatures (FIG. 3). Peel strength was found to vary strongly with bonding temperature; temperatures below 100° C. or above 200° C. were near the lower limit of detection for peel strength. Peel strength increased nearly linearly with temperatures from 100° C. to a maximum of around 0.7 kN/m at a bonding temperature of 170° C., and then fell rapidly with temperatures from 170° C. to 200° C.

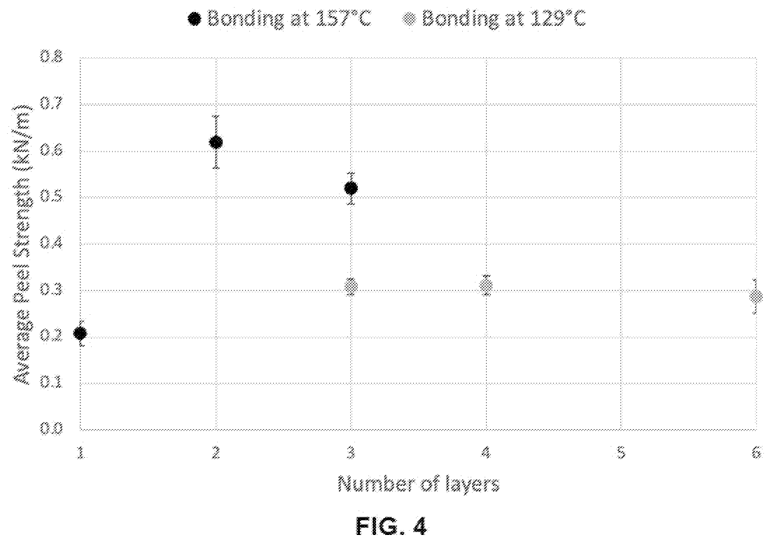

To test the dependence of peel strength on adhesive-film thickness, bonding was attempted at two intermediate temperatures (129° C. and 157° C.) with different numbers of adhesive layers applied (FIG. 4). A maximum peel strength was found using two layers of adhesive film (corresponding to approximately 100-micron total adhesive thickness). Thinner adhesive preparations were substantially weaker, while thicker preparations would presumably require higher bonding temperatures or longer treatment times to impart the required flow behavior for adhesion.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. An adhesive composition comprising one or more polypeptides, a solvent, a denaturing agent, and a plasticizer, wherein the one or more polypeptides comprise a peptide of Formula I: 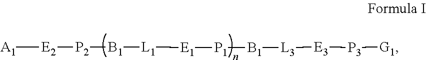
wherein
A₁ is absent, is a methionine, or is an amino acid sequence 1 to 4 residues in length;
E₁ a GLY-rich amino acid sequence 8 to 58 residues in length comprising amino acids selected from the group consisting of glycine, leucine, tyrosine, phenylalanine, and proline, or any combination thereof;
B₁ is an ASTVH-rich sequence amino acid sequence 6 to 17 residues in length comprising amino acids selected from the group consisting of alanine, serine, threonine, valine, histidine, glycine, glutamine, and proline, or any combination thereof.
L₁ is absent or is an amino acid sequence 1 to 7 residues in length comprising amino acids selected from the group consisting of proline, glycine, leucine, serine, and threonine, or any combination thereof;
E₂ is absent or is E₁;
E₃ is absent or is E₁;
P₁ is absent or is proline;
P₂ is absent or is P₁;
P₃ is absent or is P₁;
L₃ is absent or is L₁;
G₁ is absent or is an amino acid sequence 1 to 4 residues in length; and
wherein n is 4 to 100.

2. The adhesive composition of claim 1, wherein, in the one or more peptides, E₂ and E₃ are absent and L₃ is absent.

3. (canceled)

4. The adhesive composition of claim 1, wherein the solvent comprises DMSO, aqueous formic acid, neat formic acid, aqueous solutions of ammonia, an alkylamine, hexafluoroisopropanol (HFIP), or any combination thereof.

5.-6. (canceled)

7. The adhesive composition of claim 1, wherein the denaturing agent comprises an acid, an alkali, a salt, urea, an alcohol, a guanidinium salt, or any combination thereof.

8. The adhesive composition of claim 7, wherein the acid comprises hydrochloric acid.

9. The adhesive composition of claim 7, wherein the alkali comprises sodium hydroxide, potassium hydroxide, or any combination thereof.

10. The adhesive composition of claim 7, wherein the salt comprises calcium chloride, lithium chloride, or any combination thereof.

11. The adhesive composition of claim 7, wherein the alcohol comprises ethanol.

12. (canceled)

13. The adhesive composition of claim 1, wherein the plasticizer comprises water, glycerol, 1,4-butanediol, dibutyl tartrate, dibutyl phthalate, lactic acid, octanoic acid, palmitic acid, sorbitol, sucrose, diacetyl tartaric acid ester of monodiglycerides, or any combination thereof.

14. (canceled)

15. The adhesive composition of claim 1, wherein the one or more polypeptides are present in an amount of from about 0.01 to about 50% w/v.

16. The adhesive composition of claim 15, wherein the solvent is present in an amount of from about 70% to about 99.9% v/v.

17. The adhesive composition of claim 1, wherein the denaturing agent is present in an amount of from about 0.01 to about 50% w/v.

18. The adhesive composition of claim 17, wherein the plasticizer is present in an amount of from about 0.001% to about 30% v/v.

19. The adhesive composition of claim 1, wherein the composition is in the form of a film, a gel, a bandage, a nanotube, a micropattern, a ribbon, a colloid, a lithographic pattern, or a strip.

20. (canceled)

21. The adhesive composition of claim 1, wherein the composition is transparent.

22. (canceled)

23. The adhesive composition of claim 1, wherein the ASTVH-rich sequence comprises at least one alanine, at least one serine, at least one threonine, at least one valine, and at least one histidine.

24. The adhesive composition of claim 23, wherein the ASTVH-rich sequence comprises one or more of SEQ ID NOs. 1-103.

25. (canceled)

26. The adhesive composition of claim 1, wherein the GLY-rich sequence comprises at least one glycine, at least one leucine, and at least one tyrosine.

27. The adhesive composition of claim 26, wherein the GLY-rich sequence comprises one or more of SEQ ID NOs. 119-235.

28.-31. (canceled)

32. The adhesive composition of claim 1, wherein the one or more polypeptides exhibit self-healing behavior.

33.-62. (canceled)


SEQUENCE LISTING (242 sequences)

SEQ ID NO: 1 - protein, 10 aa, synthetic construct
AAAAAAAVHH

SEQ ID NO: 2 - protein, 10 aa, synthetic construct
AAAALPAVHH

SEQ ID NO: 3 - protein, 11 aa, Euprymna scolopes
AAAASVSTVHH

SEQ ID NO: 4 - protein, 10 aa, synthetic construct
AAAPLSTVHH

SEQ ID NO: 5 - protein, 10 aa, synthetic construct
AAAPVAPVHH

SEQ ID NO: 6 - protein, 11 aa, Euprymna scolopes
AAASTVSTVHH

SEQ ID NO: 7 - protein, 10 aa, Euprymna scolopes
AAASVSTAHH

SEQ ID NO: 8 - protein, 10 aa, Loligo vulgaris
AAASVSTVHH

SEQ ID NO: 9 - protein, 10 aa, synthetic construct
AAASVSTVHH

SEQ ID NO: 10 - protein, 11 aa, Loligo pealeii
AAAVSHTTHHA

SEQ ID NO: 11 - protein, 11 aa, Todarodes pacificus
AAAVSHVTHHA

SEQ ID NO: 12 - protein, 10 aa, Todarodes pacificus
AASAVSTSTH

SEQ ID NO: 13 - protein, 14 aa, Loligo vulgaris
AASSVTHTTHGVAH

SEQ ID NO: 14 - protein, 11 aa, Todarodes pacificus
AATAVHTTHHA

SEQ ID NO: 15 - protein, 10 aa, Loligo vulgaris
AATAVSHNSS

SEQ ID NO: 16 - protein, 14 aa, Loligo vulgaris
AATAVSHTTHGIHH

SEQ ID NO: 17 - protein, 11 aa, Loligo vulgaris
AATAVSHTTHH

SEQ ID NO: 18 - protein, 12 aa, Loligo vulgaris
AATAVSHTTHHA

SEQ ID NO: 19 - protein, 12 aa, Todarodes pacificus
AATAVSHVTHHA

SEQ ID NO: 20 - protein, 12 aa, Euprymna scolopes
AATSFSHTAHHA

SEQ ID NO: 21 - protein, 13 aa, Loligo vulgaris
AATSVKTVSHGFH

SEQ ID NO: 22 - protein, 13 aa, Loligo pealeii
AATSVKTVSHGYH

SEQ ID NO: 23 - protein, 14 aa, Loligo vulgaris
AATSVSHTTHGVHH

SEQ ID NO: 24 - protein, 12 aa, Todarodes pacificus
AATSVSHTTHHA

SEQ ID NO: 25 - protein, 12 aa, Loligo pealeii
AATSVSHTTHSV

SEQ ID NO: 26 - protein, 12 aa, Todarodes pacificus
AATSVSRTTHHA

SEQ ID NO: 27 - protein, 11 aa, Euprymna scolopes
AATSYSHALHH

SEQ ID NO: 28 - protein, 9 aa, Loligo vulgaris
AATTAVTHH

SEQ ID NO: 29 - protein, 11 aa, Loligo vulgaris
AATTAVTQTHH

SEQ ID NO: 30 - protein, 12 aa, Loligo pealeii
AATTVSHSTHAV

SEQ ID NO: 31 - protein, 14 aa, Loligo pealeii
AATTVSHTTHGAHH

SEQ ID NO: 32 - protein, 12 aa, Todarodes pacificus
AATTVSRTTHHA

SEQ ID NO: 33 - protein, 11 aa, Loligo vulgaris
AATTYRQTTHH

SEQ ID NO: 34 - protein, 12 aa, Euprymna scolopes
AATTYSHTAHHA

SEQ ID NO: 35 - protein, 11 aa, Euprymna scolopes
AATYSHTTHHA

SEQ ID NO: 36 - protein, 14 aa, Loligo pealeii
AGSSISTVSHGVHA

SEQ ID NO: 37 - protein, 11 aa, Todarodes pacificus
ASSAVSHTSHH

SEQ ID NO: 38 - protein, 12 aa, Loligo vulgaris
ASTSVSHTTHSV

SEQ ID NO: 39 - protein, 12 aa, Todarodes pacificus
ATAAVSHTTHHA

SEQ ID NO: 40 - protein, 14 aa, Loligo pealeii
ATASVSHTTHGVHH

SEQ ID NO: 41 - protein, 11 aa, Loligo vulgaris
ATAVSHTTHHA

SEQ ID NO: 42 - protein, 11 aa, Loligo vulgaris
AVGHTTVTHAV

SEQ ID NO: 43 - protein, 12 aa, Loligo pealeii
AVRHHTTYTHAY

SEQ ID NO: 44 - protein, 11 aa, Loligo pealeii
AVRHTTVTHAV

SEQ ID NO: 45 - protein, 9 aa, Loligo pealeii
AVSHVTHTI

SEQ ID NO: 46 - protein, 11 aa, Loligo pealeii
AVSHVTHTIHA

SEQ ID NO: 47 - protein, 15 aa, Loligo vulgaris
AVSTVSHGLGYGLHH

SEQ ID NO: 48 - protein, 11 aa, Loligo vulgaris
GAAAYSHTVHH

SEQ ID NO: 49 - protein, 6 aa, Loligo pealeii
GAAFHY

SEQ ID NO: 50 - protein, 12 aa, Loligo pealeii
GATTYSHTTHAV

SEQ ID NO: 51 - protein, 10 aa, Loligo pealeii
GHAVTHTVHH

SEQ ID NO: 52 - protein, 16 aa, Euprymna scolopes
GLLGAAATTYKHTTHH

SEQ ID NO: 53 - protein, 17 aa, Euprymna scolopes
GLLGAAATTYKHTTHHA

SEQ ID NO: 54 - protein, 11 aa, Loligo pealeii
GVAAYSHSVHH

SEQ ID NO: 55 - protein, 11 aa, Loligo pealeii
HASTTTHSIGL

SEQ ID NO: 56 - protein, 11 aa, Todarodes pacificus
HAVGAVSTLHH

SEQ ID NO: 57 - protein, 11 aa, Loligo pealeii
HAVSTVAHGIH

SEQ ID NO: 58 - protein, 13 aa, Loligo pealeii
HIGTSVSSVSHGA

SEQ ID NO: 59 - protein, 9 aa, Todarodes pacificus
HSVAVGVHH

SEQ ID NO: 60 - protein, 9 aa, Loligo pealeii
HSVSHVSHG

SEQ ID NO: 61 - protein, 10 aa, Loligo pealeii
HSVSTVSHGA

SEQ ID NO: 62 - protein, 9 aa, Loligo vulgaris
HTVSHVSHG

SEQ ID NO: 63 - protein, 12 aa, Loligo vulgaris
HVGTSVHSVSHG

SEQ ID NO: 64 - protein, 13 aa, Loligo vulgaris
HVGTSVHSVSHGV

SEQ ID NO: 65 - protein, 14 aa, Loligo vulgaris
MSSSVSHVSHTAHS

SEQ ID NO: 66 - protein, 10 aa, Loligo vulgaris
RSVSHTTHSA

SEQ ID NO: 67 - protein, 14 aa, Loligo pealeii
SAGGTTVSHSTHGV

SEQ ID NO: 68 - protein, 10 aa, Todarodes pacificus
SATAVSHTSH

SEQ ID NO: 69 - protein, 14 aa, Loligo pealeii
SHGVSHTAGYSSHY

SEQ ID NO: 70 - protein, 17 aa, Loligo pealeii
SSYYGRSASTVSHGTHY

SEQ ID NO: 71 - protein, 9 aa, Loligo pealeii
SVATRRVVY

SEQ ID NO: 72 - protein, 15 aa, Loligo vulgaris
TGASVNTVSHGISHA

SEQ ID NO: 73 - protein, 12 aa, Loligo pealeii
TGASVSTVSHGL

SEQ ID NO: 74 - protein, 12 aa, Loligo vulgaris
TGSSISTVSHGV

SEQ ID NO: 75 - protein, 14 aa, Loligo pealeii
TGSSISTVSHGVHS

SEQ ID NO: 76 - protein, 12 aa, Loligo pealeii
TGTSVSTVSHGV

SEQ ID NO: 77 - protein, 12 aa, Loligo pealeii
TSVSQVSHTAHS

SEQ ID NO: 78 - protein, 13 aa, Loligo vulgaris
VAHHGTISRRYAI

SEQ ID NO: 79 - protein, 13 aa, Todarodes pacificus
VAHHSVVSRRYAI

SEQ ID NO: 80 - protein, 14 aa, Todarodes pacificus
VASSVSHTTHGVHH

SEQ ID NO: 81 - protein, 12 aa, Todarodes pacificus
VATSVSRTTHHA

SEQ ID NO: 82 - protein, 12 aa, Todarodes pacificus
VATVTSQTSHHV

SEQ ID NO: 83 - protein, 12 aa, Todarodes pacificus
VGAAVSHVTHHA

SEQ ID NO: 84 - protein, 10 aa, Loligo vulgaris
VGAAVSTVHH

SEQ ID NO: 85 - protein, 14 aa, Loligo vulgaris
VGASVSTVSHGIGH

SEQ ID NO: 86 - protein, 14 aa, Loligo pealeii
VGASVSTVSHGVHA

SEQ ID NO: 87 - protein, 10 aa, Loligo pealeii
VGGAVSTVHH

SEQ ID NO: 88 - protein, 14 aa, Loligo vulgaris
VGQSVSTVSHGVHA

SEQ ID NO: 89 - protein, 14 aa, Loligo vulgaris
VGSTISHTTHGVHH

SEQ ID NO: 90 - protein, 15 aa, Loligo pealeii
VGSTSVSHTTHGVHH

SEQ ID NO: 91 - protein, 15 aa, Loligo pealeii
VIHGGATLSTVSHGV

SEQ ID NO: 92 - protein, 15 aa, Loligo pealeii
VIHGGATLSTVSHGV

SEQ ID NO: 93 - protein, 10 aa, Loligo pealeii
VRYHGYSIGH

SEQ ID NO: 94 - protein, 14 aa, Loligo vulgaris
VSSSVSHVSHGAHY

SEQ ID NO: 95 - protein, 13 aa, Loligo vulgaris
VSSVRTVSHGLHH

SEQ ID NO: 96 - protein, 13 aa, Loligo pealeii
VSSVSTVSHGLHH

SEQ ID NO: 97 - protein, 13 aa, Loligo vulgaris
VTHYSHVSHDVHQ

SEQ ID NO: 98 - protein, 9 aa, Loligo vulgaris
VTSAVHTVS

SEQ ID NO: 99 - protein, 9 aa, Loligo vulgaris
VVSHVTHTI

SEQ ID NO: 100 - protein, 9 aa, unidentified
YPLGHAVHR

SEQ ID NO: 101 - protein, 7 aa, unidentified
YPVVLPR

SEQ ID NO: 102 - protein, 15 aa, Loligo vulgaris
YYRKSVSTVSHGAHY

SEQ ID NO: 103 - protein, 15 aa, Loligo vulgaris
YYRRSFSTVSHGAHY

SEQ ID NO: 104 - protein, 294 aa, synthetic construct
MAAASVSTVHHPSTGTLSYGFGGLYGGLFGGLGFGPAAASVSTVHHPSTGTLSYGFGGLYGGLFGGLGFGPAAASVSTVHHPSTGTLSYGFGGLYGGLFGGLGFGPAAASVSTVHHPSTGTLSYGFGGLYGGLFGGLGFGPAAASVSTVHHPSTGTLSYGFGGLYGGLFGGLGFGPAAASVSTVHHPSTGTLSYGFGGLYGGLFGGLGFGPAAASVSTVHHPSTGTLSYGFGGLYGGLFGGLGFGPAAASVSTVHHPSTGTLSYGFGGLYGGLFGGLGFGPAAASVSTVHHPTS

SEQ ID NO: 105 - protein, 325 aa, Loligo vulgaris
MAAGHLIVSALLAVIALSSYVNAQIPGAVGGLFGAGHHLGTSYSAISHAHPVGGWGYGGGYGLGGAYGGYGLGGHTVSHVSHGPVGMGYGGLYGHYGGYGLGGAYGHGIGGVYGHGIGGLGGVYGHGIGGVYGHGIGGLYGHGFGGAYGGYGGYGIGGAVSHYGVGHRTINQVSHRYHPLGYGITYPIGHAVHRVTQPVAHHGTISRRYAIPHYEVRYVNYPVVNRYIQMVTVNQPYVVPRYETHIRSYQVPVPRYQVHMAQYPVVIPRVHIAESHHPVTHYSHVSHDVHQPIYGVHYPMGHTVESVSHHQPYVYGATHGIYGGY

SEQ ID NO: 106 - protein, 195 aa, Loligo vulgaris
MAAKLITLLAVIALSNYAYALLPGLLGGYGYPAATTYRQTTHHGYGGLYGGLGYHYPAATAVSHTTHHAPYGYGGLYGGLYGGLGYPAAASVSTVHHPVGYGGYGLGAYGAYGLGYGLHYPAATAVSHTTHHAPYGYGGLYGGLYGGLGAVSTVSHGLGYGLHHPVGYAGYGLGATAVSHTTHHAPYGGFGYGLY

SEQ ID NO: 107 - protein, 123 aa, Loligo vulgaris
MAVFIIQQPQPLATQLITPVGLGYGGLYGGLHYPVGAAVSTVHHPVGYGGFGLGFGGLYGGLHYPAATAVSHTTHHPVGLGYGGFGLGYGGLYGGFGYPGAAAYSHTVHHPVAYGGLGYGFGF

SEQ ID NO: 108 - protein, 493 aa, Loligo pealeii
MGAMQLAVALIVLGISSSANAVFNGSWVGLGSQPSPLIGKSVSTVSHGYHYPGYGGWGYGLGGWGHGLGGLGSYGLHYPMSSSVSHVSHTAHAPLGFSGWGGYAQHSPLTHVSRTALPPVGWAFGGIYRGHGAQVSQSPVRYHGYSLGHPSVATRRVVYPKPAVSHVTHTIPHADYGVSGLGGYVSSYPTGASINTVSHGISHAPVYGGWGVGFPAGQAMSTVAHGIHPTVPYGGIGLGGLYGGYGAHFPAATSVSHTTHSVPHSVGWGLGGWGGYGLGYGVHAPVGASVSTVSHGVHAPVIHGGATLSTVSHGVPALGAYGGYGFGGIVGGHSVYHPTGTSVSTVSHGVPALGGYGEYGLGGIVGGHSVSTVSHGAPALGAYGGYGLGGLVGGFGAYHPAGSSISTVSHGVHAPVGFGGYGLGGYGLGGYGLGGYGLGGYGLGGVVGGFGGYHPVGSSVSTVSHGAFGGHGLLGGYGVPLPLSTTSHHTVTH

SEQ ID NO: 109 - protein, 559 aa, Loligo vulgaris
MGAMQLAVALIVLGISSSANGIFNGSTAGLGSQPSPYIGRSVSTVSHGSHYPAYGGWGYNLGSWGHGLGGLGSYGLHYPMSSSVSHVSHTAHSPLGYYGWGGYGQQSPLTHVSRTALPPVGWAFGGLYRGHGAQVSQSPVRYHGYSFGRPAVATRRVLYPKPVVSHVTHTIPHSGWGMGGFGGYVSSYPTGASVNTVSHGISHAPVYGGWGAGHAISTVAHGIHPTVTYGGMGLGGLYGGYGAHYPASTSVSHTTHSVPHTVGLGLGSLHGGWGGYGIGYGVHSPVGASVSTVSHGIGHPVGYGTWGLGSGAHYPVGQSVSTVSHGVHAPVVHGGLGLSGSSVSTVSHGVPSLGAYGGYGLGGLIGGHSVYHPTGSSISTVSHGVPSLGAYGGYGLGGLIGGHSVYHPTGSSISTVSHGVPSLGAYGGYGLGGLIGGHSVYHPTGSSISTVSHGVPSLGAYGGYGLGGIVGGYGAYNPTGSSISTVSHGVHSPVGYGGYGLGGLIGGYGAYHPAGSSISTVSHGINSLGAYGGYGNGGLLGGYGVPLPLSTTSHHTVTH

SEQ ID NO: 110 - protein, 384 aa, Loligo vulgaris
MGAMQLAVALIVLGISSSANGIFNGSTAGLGSQPSPYIGRSVSTVSHGSHYPAYGGWGYNLGSWGHGLGGLGSYGLHYPMSSSVSHVSHTAHSPLGYYGWGGYGQQSPLTHVSRTALPPVGWAFGGLYRGHGAQVSQSPVRYHGYSFGRPAVATRRVLYPKPVVSHVTHTIPHSGWGMGGFGGYVSSYPTGASVNTVSHGISHAPVYGGWGAGHAISTVAHGIHPTVTYGGMGLGGLYGGYGAHYPASTSVSHTTHSVPHTVGLGLGSLHGGWGGYGIGYGVHSPVGASVSTVSHGIGHPVGYGTWGLGSGAHYPVGQSVSTVSHGVHAPVVHGGLGLSGSSVSTVSHGVPSLGAYGGYGNGGLLGGYGVPLPLSTTSHHTVTH

SEQ ID NO: 111 - protein, 162 aa, synthetic construct
MGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYG

SEQ ID NO: 112 - protein, 268 aa, synthetic construct
MGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGP

SEQ ID NO: 113 - protein, 443 aa, synthetic construct
MGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGPAAASVSTVHHPSTGTLSYGYGGLYGGLYGGLGYGP

SEQ ID NO: 114 - protein, 337 aa, synthetic construct
MGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPTS

SEQ ID NO: 115 - protein, 199 aa, synthetic construct
MTYGYGGLYGGLYGGLGYPAAASVSTVHHPYGYGGLYGGLYGGLGYPAAASVSTVHHPYGYGGLYGGLYGGLGYPAAASVSTVHHPYGYGGLYGGLYGGLGYPAAASVSTVHHPYGYGGLYGGLYGGLGYPAAASVSTVHHPYGYGGLYGGLYGGLGYPAAASVSTVHHPYGYGGLYGGLYGGLGYPAAASVSTVHHPS

SEQ ID NO: 116 - protein, 249 aa, Loligo vulgaris
MVATVLVIMSMIAALSCQSEAALSVGTSVKTIRQSVHHGAVPAVGHTTVTHAVPHAYAYGGLPYGDAFGGLYGGLYGGLYGSPAATSVKTVSHGFHPTLPVGSTISHTTHGVHHPVTYGGLGLGGLGYGGLGYGGLGYGGLGYGGLGYGGLGYGGLGYGGLGAGGLYGLHYPGAVGLGYGLGGGYGGLYGLHLPAATSVSHTTHGVHHPALGLGLYGGAHLPAASSVTHTTHGVAHPGLGLNYGVYGLH

SEQ ID NO: 117 - protein, 330 aa, synthetic construct
MVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPSTGTLSYGYGGLFGGLFGGLGYGPVGQSVSTVSHGVHAPTS

SEQ ID NO: 118 - protein, 330 aa, synthetic construct
MVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPSTGTLSYGYGGLYGGLYGGLGYGPVGQSVSTVSHGVHAPTS

SEQ ID NO: 119 - protein, 18 aa, Loligo pealeii
AGLGYGLGGVYGGYGLHA

SEQ ID NO: 120 - protein, 21 aa, Loligo pealeii
ALGAYGGYGFGGIVGGHSVYH

SEQ ID NO: 121 - protein, 21 aa, Loligo pealeii
ALGAYGGYGLGGLVGGFGAYH

SEQ ID NO: 122 - protein, 17 aa, Loligo pealeii
ALGEYGGYGLGGIVGGH

SEQ ID NO: 123 - protein, 16 aa, Loligo pealeii
ALGGYGGYGLGGIVGG

SEQ ID NO: 124 - protein, 21 aa, Loligo pealeii
ALGGYGGYGLGGIVGGHSVYH

SEQ ID NO: 125 - protein, 12 aa, Loligo vulgaris
ALGLGLYGGAHL

SEQ ID NO: 126 - protein, 26 aa, Loligo pealeii
AYGGWGYSLGRWGQGLGGLGTYGLHY

SEQ ID NO: 127 - protein, 24 aa, Loligo pealeii
AYGYGYGLGGYGGYGLYGGYGLHH

SEQ ID NO: 128 - protein, 28 aa, Loligo pealeii
FGYGGVGYGGLGYGGLGYGVGGLYGLQY

SEQ ID NO: 129 - protein, 21 aa, Loligo vulgaris
GAVGLGYGLGGGYGGLYGLHL

SEQ ID NO: 130 - protein, 20 aa, Loligo pealeii
GFGGYGLGGYGLGGYGLGGY

SEQ ID NO: 131 - protein, 39 aa, Todarodes pacificus
GGYGGLYGGYGLGGYGGLHGAYGLGGYGGVYGGYGLGGH

SEQ ID NO: 132 - protein, 19 aa, Todarodes pacificus
GGYGSLLGGHGGLYGGLGL

SEQ ID NO: 133 - protein, 27 aa, Loligo pealeii
GIGGAYGGYGLGVGGLYGGYGGYGIGG

SEQ ID NO: 134 - protein, 51 aa, Loligo vulgaris
GIGGVYGHGIGGLGGVYGHGIGGVYGHGIGGLYGHGFGGAYGGYGGYGIGG

SEQ ID NO: 135 - protein, 12 aa, Loligo vulgaris
GLGLGYGLGLGL

SEQ ID NO: 136 - protein, 16 aa, Loligo pealeii
GLGLGYGLGLGYGYGV

SEQ ID NO: 137 - protein, 15 aa, Loligo vulgaris
GLGLGYGYGLGHGLG

SEQ ID NO: 138 - protein, 12 aa, Loligo vulgaris
GLGLNYGVYGLH

SEQ ID NO: 139 - protein, 31 aa, Loligo vulgaris
GVGGLYGGYGLGGLHGGYGLGGIYGGYGAHY

SEQ ID NO: 140 - protein, 33 aa, Loligo vulgaris
GVGGLYGGYGLGGLHYGGYGLGGLHYGGYGLHY

SEQ ID NO: 141 - protein, 40 aa, Loligo vulgaris
GVGGLYGGYGLGGLYGGYGLGGLHGGYSLGGLYGGYGAHY

SEQ ID NO: 142 - protein, 27 aa, Loligo vulgaris
GVGGYGMGGLYGGYGLGGVYGGYGLGG

SEQ ID NO: 143 - protein, 57 aa, Loligo pealeii
GVYGLGHGAYGLGGYGGLHGAYGLGGYGGLYGGYGLGGYGALHGGLYGGYGLGGGLL

SEQ ID NO: 144 - protein, 13 aa, Loligo vulgaris
GYGGLYGGLGYHY

SEQ ID NO: 145 - protein, 20 aa, Loligo vulgaris
GYGGLYGHYGGYGLGGAYGH

SEQ ID NO: 146 - protein, 27 aa, Loligo pealeii
GYGGVGYGGLGYGGLGYGVGGLYGLQY

SEQ ID NO: 147 - protein, 20 aa, Loligo vulgaris
GYGGWGYGLGGWGHGLGGLG

SEQ ID NO: 148 - protein, 26 aa, Loligo pealeii
GYGGWGYGLGGWGHGLGGLGSYGLHY

SEQ ID NO: 149 - protein, 18 aa, Loligo pealeii
GYGGYGLGFGGLYGGFGY

SEQ ID NO: 150 - protein, 18 aa, Loligo pealeii
GYGGYGLGLGGLYGGLHY

SEQ ID NO: 151 - protein, 35 aa, Loligo vulgaris
GYGLGGLYGGYGLGGLHYGGYGLGGLHYGGYGLHY

SEQ ID NO: 152 - protein, 15 aa, Loligo pealeii
GYGLGLGLGGAGYGY

SEQ ID NO: 153 - protein, 13 aa, Loligo pealeii
GYGLGLGLYGAHL

SEQ ID NO: 154 - protein, 15 aa, Loligo pealeii
GYGLGLGYGLGLGAG

SEQ ID NO: 155 - protein, 8 aa, Loligo vulgaris
GYGLGVGL

SEQ ID NO: 156 - protein, 16 aa, Loligo pealeii
GYGYGGLLGGLGHAVG

SEQ ID NO: 157 - protein, 26 aa, Todarodes pacificus
GYGYGGLLGGYGGLYGGWGGVYGGLG

SEQ ID NO: 158 - protein, 15 aa, Loligo pealeii
GYGYGGLLGGYGLHY

SEQ ID NO: 159 - protein, 16 aa, Loligo pealeii
HADYGVSGLGGYVSSY

SEQ ID NO: 160 - protein, 14 aa, Loligo pealeii
HAVGFGGWGHGFGY

SEQ ID NO: 161 - protein, 17 aa, Loligo pealeii
HLGLGLGYGYGLGHGLG

SEQ ID NO: 162 - protein, 21 aa, Loligo pealeii
HSVGWGLGGWGGYGLGYGVHA

SEQ ID NO: 163 - protein, 21 aa, Loligo pealeii
HSVGWGLGGWGGYGLGYGVRS

SEQ ID NO: 164 - protein, 19 aa, Loligo pealeii
HSVSYGGWGFGHGGLYGLH

SEQ ID NO: 165 - protein, 33 aa, Loligo pealeii
IGFGGWGHGYGYSGLGFGGWGHGLGGWGHGYGY

SEQ ID NO: 166 - protein, 48 aa, Loligo pealeii
LFGGILGGYGGVLAGYGGLHHGAYGLGGYGGLYGGYGLGGYGLHGLHY

SEQ ID NO: 167 - protein, 42 aa, Loligo pealeii
LGFGGLLGGYGGLHHGVYGLGGYGGLYGGYGLGGYGLHGLHY

SEQ ID NO: 168 - protein, 14 aa, Loligo pealeii
LGFGGVLGYGGLHH

SEQ ID NO: 169 - protein, 52 aa, Todarodes pacificus
LGFGGVLGYGGLHHGAYGLGGYGGLYGGYGLGGYGGLYGGYGALHGGYGLGY

SEQ ID NO: 170 - protein, 57 aa, Loligo pealeii
LGFGGVLGYGGLHHGVYGLGGYGGLHGAYGLGGYGGLHGAYGLGGYGGLYGGTLSTL

SEQ ID NO: 171 - protein, 58 aa, Loligo pealeii
LGFGGVLGYGGLHHGVYGLGGYGGLHGAYGLGGYGGLHGAYGLGGYGGLYGGYGLGGH

SEQ ID NO: 172 - protein, 75 aa, Loligo pealeii
LGFGGVLGYGGLHHGVYGLGGYGGLHGAYGLGGYGGLHGAYGLGGYGGLYGGYGLGGYGALHGGLYGGYGLGGGL

SEQ ID NO: 173 - protein, 46 aa, Todarodes pacificus
LGFGGVLGYGGLHHGVYGLGGYGGLHGAYGLGGYGGLYGGYGLGGH

SEQ ID NO: 174 - protein, 47 aa, Todarodes pacificus
LGFGGVLGYGGLHHGVYGLGGYGGLHGAYGLGGYGLGGFHGGYGLGG

SEQ ID NO: 175 - protein, 28 aa, Loligo vulgaris
LGLGYGGYGLGLGYGLGHGYGLGLGAGI

SEQ ID NO: 176 - protein, 21 aa, Loligo pealeii
LGLGYGYGLGLGYGLGLGAGI

SEQ ID NO: 177 - protein, 28 aa, Todarodes pacificus
LGYGGLLGGYGALHGGLYGGYGLGGLHY

SEQ ID NO: 178 - protein, 19 aa, Todarodes pacificus
LGYGGLLGGYGGLHHGVYG

SEQ ID NO: 179 - protein, 26 aa, Todarodes pacificus
LGYGGLLGGYGGLYGGYGLGGYGLGY

SEQ ID NO: 180 - protein, 26 aa, Todarodes pacificus
LGYGGLLGGYGGLYGRYGVGGYGLGY

SEQ ID NO: 181 - protein, 38 aa, Loligo pealeii
LGYGLAGYGGLYGGLYGGHGLGGYGGVYGGYGLHGLHY

SEQ ID NO: 182 - protein, 30 aa, Loligo vulgaris
MGGLYGGYGLGGVYGGYGLGGIYGGYGAHY

SEQ ID NO: 183 - protein, 21 aa, Loligo vulgaris
SLGAYGGYGLGGIVGGYGAYN

SEQ ID NO: 184 - protein, 21 aa, Loligo vulgaris
SLGAYGGYGLGGLIGGHSVYH

SEQ ID NO: 185 - protein, 21 aa, Todarodes pacificus
TALGYGGLYGGYGLGAYGLGY

SEQ ID NO: 186 - protein, 31 aa, Loligo pealeii
VAYGGLGLGALGYGGLGYGGLGAGGLYGLHY

SEQ ID NO: 187 - protein, 35 aa, Loligo pealeii
VAYGGLGLGALGYGGLGYGGLGYGGLGAGGLYGLH

SEQ ID NO: 188 - protein, 12 aa, Loligo vulgaris
VAYGGLGYGFGF

SEQ ID NO: 189 - protein, 41 aa, Loligo vulgaris
VAYGGWGYGLGGLHGGWGYGLGGLHGGWGYALGGLYGGLHY

SEQ ID NO: 190 - protein, 29 aa, Loligo pealeii
VAYGGWGYGLGGLHGGWGYGLGGLYGGLH

SEQ ID NO: 191 - protein, 39 aa, Loligo pealeii
VGFGGYGLGGYGLGGYGLGGYGLGGYGLGGLVGGYGSYH

SEQ ID NO: 192 - protein, 39 aa, Loligo pealeii
VGFGGYGLGGYGLGGYGLGGYGLGGYGLGGVVGGFGGYH

SEQ ID NO: 193 - protein, 38 aa, Todarodes pacificus
VGFGGYGYGGIGGLYGGHYGGYGLGGAYGHYGGYGLGG

SEQ ID NO: 194 - protein, 19 aa, Loligo pealeii
VGGYGGFGLGGYGGYGLGG

SEQ ID NO: 195 - protein, 27 aa, Todarodes pacificus
VGLGLGGFHGGYGFGGYGLGGFHGGYG

SEQ ID NO: 196 - protein, 21 aa, Loligo vulgaris
VGLGYGGFGLGYGGLYGGFGY

SEQ ID NO: 197 - protein, 14 aa, Loligo vulgaris
VGLGYGGLYGGLHY

SEQ ID NO: 198 - protein, 14 aa, Loligo pealeii
VGWGLGGLYGGLHH

SEQ ID NO: 199 - protein, 9 aa, Loligo vulgaris
VGYAGYGLG

SEQ ID NO: 200 - protein, 50 aa, Loligo pealeii
VGYAGYGLGAYGGYAGYGLGAFGGYAGYGLGAFGGYAGLGLGLYGAGYHY

SEQ ID NO: 201 - protein, 17 aa, Loligo pealeii
VGYAGYGLGLYGAGYHY

SEQ ID NO: 202 - protein, 30 aa, Loligo pealeii
VGYAGYGYGLGAYGGYAGLGLGLYGAGYHY

SEQ ID NO: 203 - protein, 30 aa, Loligo pealeii
VGYAGYGYGLGSYGGYAGLGLGLYGAGYHY

SEQ ID NO: 204 - protein, 15 aa, Loligo pealeii
VGYGGFGLAGYGYGY

SEQ ID NO: 205 - protein, 19 aa, Loligo vulgaris
VGYGGFGLGFGGLYGGLHY

SEQ ID NO: 206 - protein, 58 aa, Loligo pealeii
VGYGGLYGHYGGYGLGGVYGHGVGLGGVYGHGIGGAYGGYGLGVGGLYGGYGGYGIGG

SEQ ID NO: 207 - protein, 41 aa, Loligo pealeii
VGYGGLYGHYGGYGLGGVYGHGVGLGGVYGHGVGLGGVYSH

SEQ ID NO: 208 - protein, 22 aa, Loligo vulgaris
VGYGGYGLGAYGAYGLGYGLHY

SEQ ID NO: 209 - protein, 25 aa, Loligo pealeii
VGYGGYGLGGYGGYGLGGLTGGYGV

SEQ ID NO: 210 - protein, 33 aa, Todarodes pacificus
VGYGGYGYGGGLYGGHYGGYGHFGGVHSHYGVG

SEQ ID NO: 211 - protein, 30 aa, Todarodes pacificus
VGYGGYGYGGLGAYGHYGGYGLGGLYGGYG

SEQ ID NO: 212 - protein, 58 aa, Loligo pealeii
VGYGGYGYGGLGAYGHYGGYGLGGLYGGYGLGGAYGGYGLGGGYGGYGVGVHSRYGVG

SEQ ID NO: 213 - protein, 21 aa, Loligo pealeii
VGYGYGGFLGGYGLGVYGHGY

SEQ ID NO: 214 - protein, 27 aa, Loligo pealeii
VGYGYGGLLGGYGGLYGGWGGVYGGLG

SEQ ID NO: 215 - protein, 19 aa, Loligo pealeii
VLSGGLGLSGLSGGYGTYR

SEQ ID NO: 216 - protein, 53 aa, Loligo vulgaris
VTYGGLGLGGLGYGGLGYGGLGYGGLGYGGLGYGGLGYGGLGYGGLGAGGLYG

SEQ ID NO: 217 - protein, 10 aa, Todarodes pacificus
VYGGYGLGGH

SEQ ID NO: 218 - protein, 17 aa, Loligo pealeii
YAYGGLYGGYGLGAYGY

SEQ ID NO: 219 - protein, 19 aa, Loligo pealeii
YGDVYGGLYGGLYGGLLGA

SEQ ID NO: 220 - protein, 17 aa, synthetic construct
YGFGGLYGGLFGGLGFG

SEQ ID NO: 221 - protein, 9 aa, Loligo vulgaris
YGGFGYGLY

SEQ ID NO: 222 - protein, 17 aa, Loligo pealeii
YGGIGLGGLYGGYGAHF

SEQ ID NO: 223 - protein, 15 aa, Todarodes pacificus
YGLAGYGGLYGGLLH

SEQ ID NO: 224 - protein, 17 aa, synthetic construct
YGYGGLFGGLFGGLGYG

SEQ ID NO: 225 - protein, 29 aa, Loligo pealeii
YGYGGLYGGLGYHAGYGLGGYGLGYGLHY

SEQ ID NO: 226 - protein, 15 aa, Loligo vulgaris
YGYGGLYGGLYGGLG

SEQ ID NO: 227 - protein, 16 aa, Loligo vulgaris
YGYGGLYGGLYGGLGY

SEQ ID NO: 228 - protein, 17 aa, synthetic construct
YGYGGLYGGLYGGLGYG

SEQ ID NO: 229 - protein, 24 aa, Loligo pealeii
YGYGGLYGGYAGLGLGLYGAGYHY

SEQ ID NO: 230 - protein, 13 aa, Todarodes pacificus
YGYGGVLGGYGQG

SEQ ID NO: 231 - protein, 29 aa, Loligo pealeii
YGYGLAGYGGLYGGLHGAAYGLGGYGLHY

SEQ ID NO: 232 - protein, 17 aa, Todarodes pacificus
YGYGLAGYGGLYGGLLH

SEQ ID NO: 233 - protein, 24 aa, Todarodes pacificus
YGYGLAGYGGLYGGYGLGGYGLGY

SEQ ID NO: 234 - protein, 15 aa, Loligo vulgaris
YIGRSVSTVSHGSHY

SEQ ID NO: 235 - protein, 17 aa, Loligo pealeii
YSYGGLVGGYGGLYHHA

SEQ ID NO: 236 - protein, 4 aa, unidentified
PSTG

SEQ ID NO: 237 - protein, 5 aa, unidentified
PSTGT

SEQ ID NO: 238 - protein, 6 aa, unidentified
PSTGTL

SEQ ID NO: 239 - protein, 7 aa, unidentified
PSTGTLS

SEQ ID NO: 240 - protein, 4 aa, unidentified
STGT

SEQ ID NO: 241 - protein, 5 aa, unidentified
STGTL

SEQ ID NO: 242 - protein, 6 aa, unidentified
STGTLS